(19) United States
(12) Patent Application Publication
(10) Pub. No.: US 2022/0322839 A1
(43) Pub. Date: Oct. 13, 2022

(54) Bed Inclining System

(71) Applicant: Slope Sleep, LLC, Orleans, MA (US)

(72) Inventors: James Bond, Rochester, NY (US); Michael Marks, Orleans, MA (US)

(73) Assignee: Slope Sleep, LLC, Orleans, MA (US)

(21) Appl. No.: 17/707,478

(22) Filed: Mar. 29, 2022

Related U.S. Application Data
(60) Provisional application No. 63/172,225, filed on Apr. 8, 2021.

(51) Int. Cl. A47C 19/04; A47C 31/00

(52) U.S. Cl. CPC A47C 19/045; A47C 31/008

(57) ABSTRACT

A system for adjusting the incline of a bed is provided. The bed inclining system includes a base frame mountable to an end of an existing bed or bed frame, wherein the base frame can support a mid-section of the bed in an inclined configuration to prevent a non-planar sleeping surface. A lifting assembly extends from the base frame and can raise and lower a bed support, such that the bed is moveable between a level configuration and a raised configuration. In the level configuration, the bed is in a horizontal position and in the raised configuration, the end of the bed is raised from a floor surface, whereas an opposite end of the bed remains on the floor surface. The bed support extends from the base frame and can support the end of the bed in both the level configuration and the inclined configuration.

### CROSS REFERENCE TO RELATED APPLICATION

This application claims the benefit of U.S. Provisional Application No. 63/172,225 filed on Apr. 8, 2021. The above identified patent application is herein incorporated by reference in its entirety.

### BACKGROUND OF THE INVENTION

The present invention relates to adjustable beds. The present invention further provides a bed inclining system configured to be secured to an existing bed to raise and lower the head of the existing bed while supporting the mid-section of the bed to prevent a non-planar sleeping surface.

Many people who suffer from gastro-esophageal reflux, congestive heart disease, sleep apnea, chronic sinusitis, edema, among other ailments, are able to alleviate and treat these medical problems by sleeping in an inclined position having their head and chest or torso in an elevated position. Known adjustable beds are designed to allow a user to raise the head of the bed, to allow their head and torso to be raised relative to the foot end of the bed. Some beds also allow a user to raise their knees and feet by adjusting the incline of the foot of the bed relative to the head of the bed. These known adjustable beds require a specifically designed frame configured to bend or angle to a pre-allowed adjustable position, which also requires a mattress specifically designed to correspond to and align with the different positions permitted by the underlying frame. Due to the pre-configured bed frame and mattress designs, the mechanism used in these known adjustable beds is unable to be used with an existing bed having a conventional mattress supporting assembly.

Other bed lifting devices are configured to only support a single end of a bed, resulting in an unsupported mid-section of the bed. This lack of structural support can lead to a non-planar sleeping surface when the weight of the sleeper is added to the bed while in the inclined position. Additional stress to the bed frame while one end is lifted, and the remaining bed frame is unsupported can lead to deformation of the bed frame at stress points.

The present invention provides to a bed inclining system adapted to be positioned beneath an end of a bed frame or other bed structure of an existing bed, such that a user can selectively raise the head or foot of the existing bed by a desired amount via a remote or adjacent control, while simultaneously allowing the opposite end of the bed to remain on a floor surface. The present invention further provides a support assembly configured to support a mid-section of the bed to prevent the mattress from forming a non-planar sleeping surface.

In light of the devices disclosed in the known art, it is submitted that the present invention substantially diverges in design elements and methods from the known art and consequently it is clear that there is a need in the art for an improvement for a bed inclining system. In this regard the instant invention substantially fulfills these needs.

### SUMMARY OF THE INVENTION

In view of the foregoing disadvantages inherent in the known types of bed inclining systems now present in the known art, the present invention provides a new bed inclining system wherein the same can be utilized for securing to an existing bed and adjusting the incline of the bed via raising or lowering a head end of the bed.

It is an objective of the present invention to provide an embodiment of the bed inclining system comprising a base frame mountable to an existing bed, wherein the base frame includes an elongated bar adapted to be positioned at a head or foot of the bed.

It is another objective of the present invention to provide an embodiment of the bed inclining system comprising an embodiment having a support structure mounted to a mid-section of the bed to support the mid-section in an inclined configuration.

It is another objective of the present invention to provide an embodiment of the bed inclining system comprising an embodiment wherein the support structure is optional and can be removably secured to the elongated bar via brackets.

It is another objective of the present invention to provide an embodiment of the bed inclining system comprising a bed support and a lifting assembly both extending from the base frame. The bed support comprises a platform configured to receive a leg of the bed thereon, wherein the lifting support is configured to raise and lower the bed support, such that the bed is moveable between a level configuration and a raised configuration.

It is another objective of the present invention to provide an embodiment of the bed inclining system wherein the level configuration, the bed is in a horizontal position and in the raised configuration, the head end of the bed is raised from a floor surface, whereas a foot end of the bed remains on the floor surface.

It is another objective of the present invention to provide an embodiment of the bed inclining system that is adapted to be entirely removable from the existing bed frame so that it can be mounted to either end of the bed frame or to a different bed frame.

It is yet another objective of the present invention to provide an embodiment of the bed inclining system wherein the lifting assembly comprises an actuator disposed within a housing. The operation of the actuator from the level configuration to the raised configuration is configured to apply a lifting force to the elongated bar of the base frame resulting in the elevation of the bed support.

It is yet another objective of the present invention to provide an embodiment of the bed inclining system comprising a remote device having an application configured to track user data, such as heart rate, respiration, snoring, and sleep position. The system is configured to automatically adjust a slope of the bed in response to the user data for a better sleep position.

It is therefore an object of the present invention to provide a new and improved bed inclining system that has all of the advantages of the known art and none of the disadvantages.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of a first embodiment of the bed inclining system.

FIG. 2 shows a perspective view of an embodiment of the bed inclining system in the raised position.

FIG. 3 shows a close-up view of the lifting assembly of an embodiment of the bed inclining system.

FIG. 4 shows a rear perspective view of a first embodiment of the bed inclining system.

FIG. 5 shows a close-up view of the bed support of an embodiment of the bed inclining system.

FIG. 6 shows a perspective view of a second embodiment of the bed inclining system in the raised position.

FIG. 7 shows a cross sectional view of a lifting assembly of an embodiment of the bed inclining system in the raised position.

FIG. 8 shows a cross sectional view of a lifting assembly of an embodiment of the bed inclining system in the lowered position.

FIG. 9 shows a perspective view of a third embodiment of the bed inclining system.

FIG. 10 shows a rear view of a third embodiment of the bed inclining system in use.

FIG. 11 shows a side view of a third embodiment of the bed inclining system in use.

FIG. 12 shows a block diagram of an embodiment of the bed inclining system

### DETAILED DESCRIPTION OF THE INVENTION

Reference is made herein to the attached drawings. For the purposes of presenting a brief and clear description of the present invention, the preferred embodiment will be discussed as used for raising and lowering an end of a bed. The figures are intended for representative purposes only and should not be considered to be limiting in any respect.

Reference will now be made in detail to the exemplary embodiment (s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment”, “an alternate embodiment”, “first embodiment”, “second embodiment”, or “third embodiment” does not necessarily refer to the same embodiment. Additionally, “bed”, “bed frame”, and “mattress support structure” may be used interchangeably, and all refer to the type of pre-existing bed that the present invention is adapted to mount.

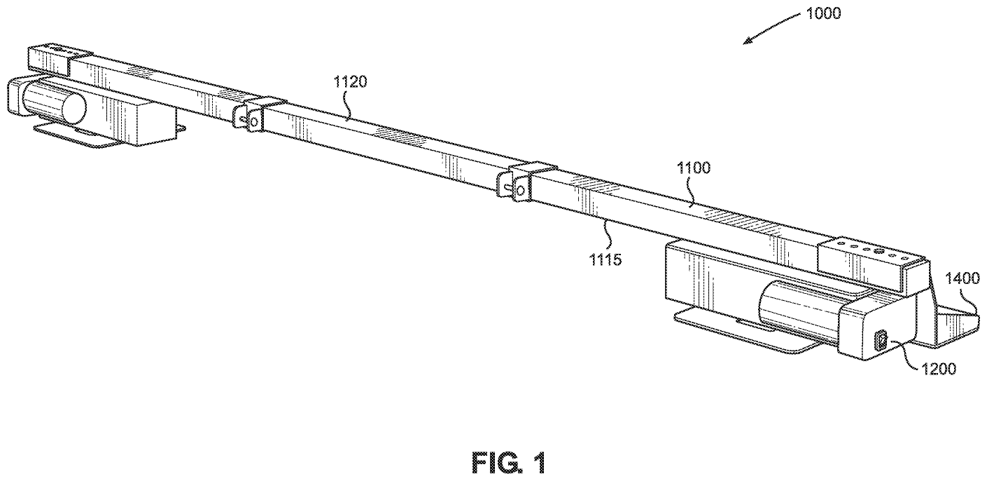

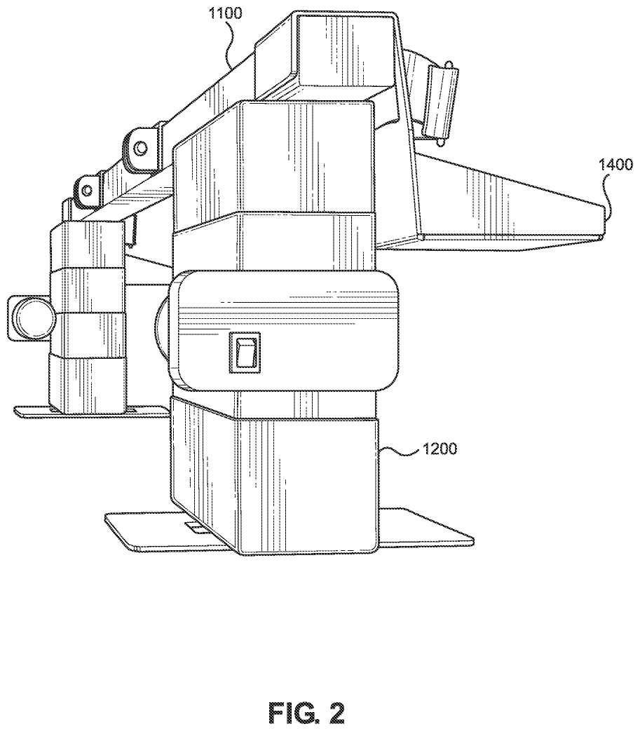

Referring now to FIGS. 1-3, there is shown a front perspective view of a first embodiment of the bed inclining system in a lowered position, a perspective view of first embodiment of the bed inclining system in the raised position, and a close-up view of the lifting assembly of an embodiment of the bed inclining system, respectively. The bed inclining system 1000 comprises a base frame 1100. The base frame 1100 is configured to be positioned at a head end of a conventional or pre-existing bed to allow the bed inclining system 1000 to raise and lower the head of the bed to a selected height wherein the length of the bed achieves a desired slope. In the illustrated embodiment, the base frame 1100 comprises an elongated, rigid bar 1115 that is adapted to be supported by a single or multiple lifting assemblies 1200, in conjunction with a bed support 1400 of the bed inclining system 1000. In the illustrated embodiment, the base frame 1100 is configured to connect multiple lifting assemblies 1200 along different positions of the elongated bar 1115 such that the lifting assemblies are securely disposed on a same horizontal plane and operate synchronically. In some embodiments, the base frame 1100 comprises a length equivalent to a width of a bed frame it is configured to mount. For example, in one embodiment, the base frame 1100 is configured to mount to a standard king size bed frame and therefore comprises a length between 75 inches to 80 inches. In alternate embodiments, the base frame 1100 is configured to mount any size bed frame and comprises any suitable length.

In some embodiments, the base frame 1100 is adjustable in length so it can mount to various sized bed frames. In the illustrated embodiment, the elongated bar 1115 is telescopic and comprises at least one telescoping segment 1120 to allow the base frame 1100 to fit under beds having different widths, wherein the width is measured between opposing lateral sides of a bed. In the illustrated embodiment, the telescoping segment 1120 is configured to fit within adjacent outer segments of the elongated bar 1115. In this way, the length of the bar 1115 is adjustable. In the illustrated embodiment, the base frame 1100 comprises a square shaped cross section. However, in alternate embodiments, the base frame comprises any suitable shape, such as having a circular or rectangular shaped cross section.

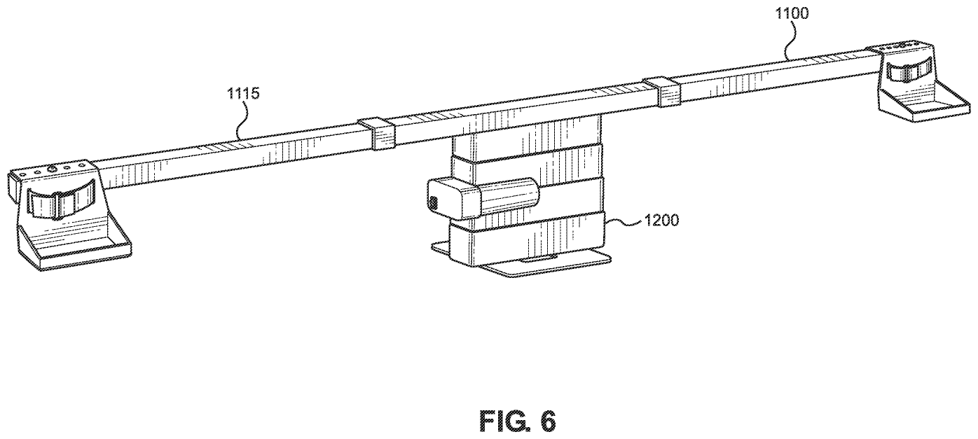

At least one lifting assembly 1200 is secured to the base frame 1100, wherein the lifting assembly 1200 comprises the components to raise and lower the bed. In the illustrated embodiment, the bed inclining system comprises a pair of lifting assemblies 1200 each disposed on opposing sides of the base frame 1100. However, in alternate embodiments, the bed inclining system 1000 comprises a single lifting assembly 1200 that can be positioned anywhere along the base frame 1100. In some embodiments, such as the embodiment shown in FIG. 6, the lifting assembly 1200 is positioned in a center of the base frame 1100. In other embodiments, more than a pair of lifting assemblies are secured to the base frame and needed to lift heavier weighted beds and to accommodate heavier users. In the illustrated embodiment, the lifting assembly 1200 comprises a local control 1210 configured to raise and lower the lifting assembly 1200.

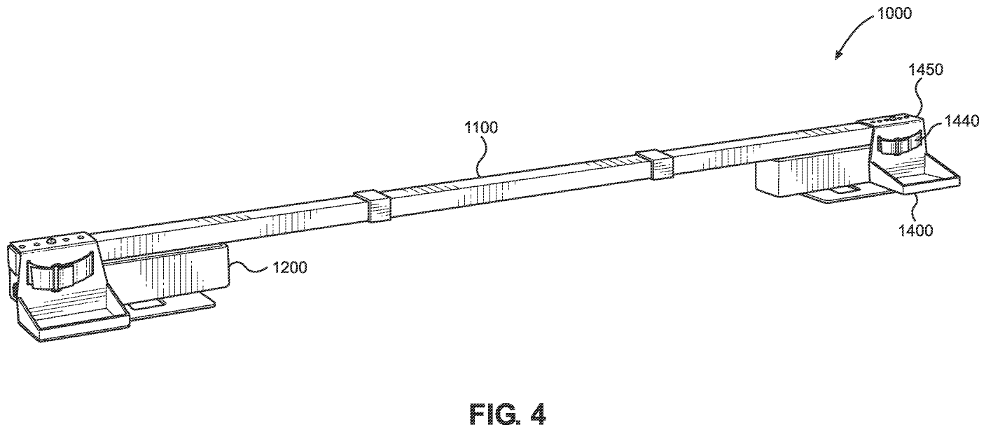

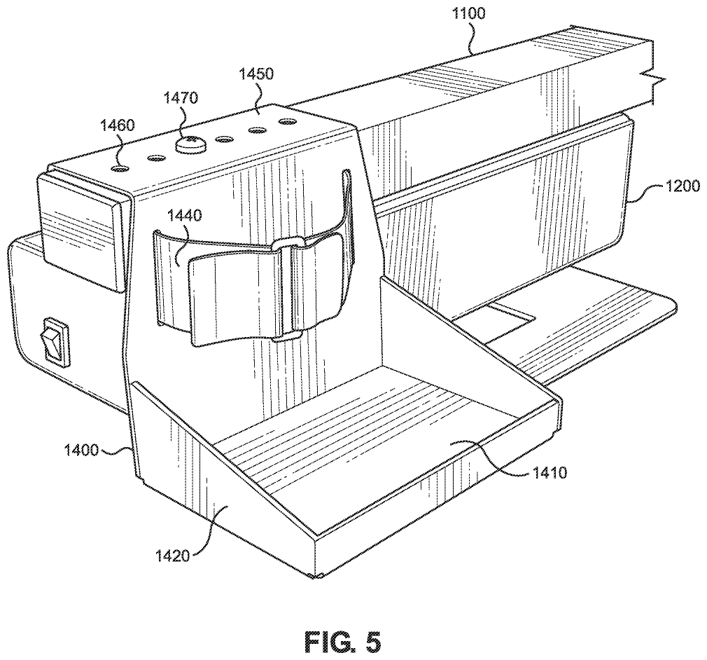

Referring now to FIGS. 4 and 5, there is shown a rear perspective view of a first embodiment of the bed inclining system and a close-up view of the bed support of an embodiment of the bed inclining system, respectively. The bed inclining system 1000 further comprises a bed support 1400 configured to receive a portion of the bed so when the lifting assembly is activated, the bed is raised or lowered via securement of the bed to the bed support 1400. In the illustrated embodiment, a pair of bed supports 1400 extend from the base frame 1100, opposite to the lifting assembly 1200, such that each bed support is configured to receive a leg of the bed. When mounted to the bed, the bed support 1400 remains on an exterior side of the base frame 1100 such that the remaining base frame and lifting assembly 1200 are concealed and positioned beneath the bed.

The bed support 1400 comprises a platform 1410 adapted to receive the leg or edge of the bed frame. In the illustrated embodiment, the platform 1410 is flat and comprises a rectangular shaped cross-section. However, in alternate embodiments, the platform comprises any suitable shape configured to receive a leg or edge of a bed thereon. In the illustrated embodiment, a sidewall 1420 is disposed along a perimeter of the platform 1410 to prevent the bed frame from sliding from the bed support 1400. In some embodiments, the sidewall 1420 extends along at least three sides of the platform 1410. In other embodiments, the bed support 1400 comprises a single sidewall extending from the outer perimeter of the platform 1410. In this way, interior sides of the platform are adapted to receive a corner of a platform style bed or bed frame as opposed to a leg. In some embodiments, the platform 1410 is absent at least one sidewall to enable it to be more easily slid beneath a bed leg or bed supporting element.

In the illustrated embodiment, each bed support 1400 is adjustably or slidably mounted to the base frame 1100 via a U-shaped plate 1450. The plate 1450 comprises a plurality of apertures 1460 that are configured to align above an aperture disposed on the base frame 1100. Once the apertures of the bed support and the base frame are aligned, a corresponding pin 1470 is disposed through each aperture to secure the bed support 1400 to the base frame 1100. In this way, a distance between the pair of bed supports is adjustable to accommodate for the legs and edges of a bed from having differing widths.

In the illustrated embodiment, the bed support 1400 comprises a fastener adapted to secure a leg of the bed frame to the base frame 1100. In the shown embodiment, the fastener is a pair of securable straps 1440 extending from the bed support 1400 and configured to form a loop around the leg. In some embodiments, the bed support does not include a fastener.

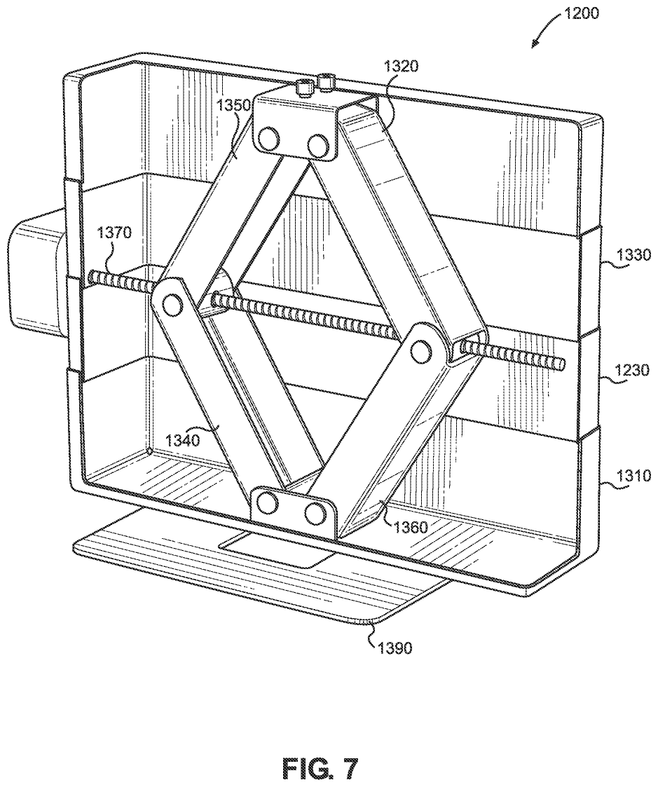

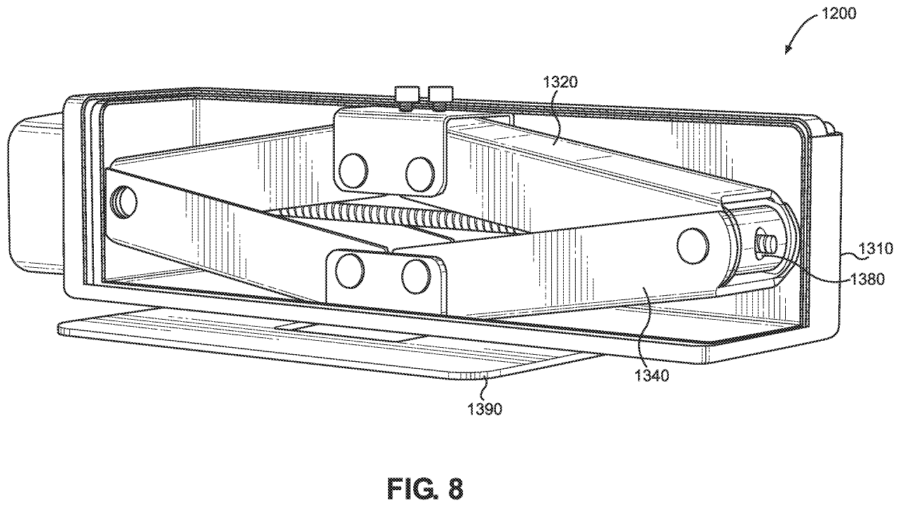

Referring now to FIGS. 7 and 8, there is shown a cross sectional view of a lifting assembly of an embodiment of the bed inclining system in the raised position and a cross sectional view of a lifting assembly of an embodiment of the bed inclining system in the lowered position, respectively. In the illustrated embodiment, the lifting assembly 1200 comprises a telescopic or vertically expandable housing 1310 secured to a lower side of the base frame and an actuator 1320 disposed within the housing 1310 adapted to extend and retract the housing 1310. The housing 1310 comprises at least one telescopic section 1330 that is retracted within the housing 1310 when the actuator 1320 is in a lowered position. In an alternative embodiment, the housing comprises an elastomeric material such as spandex or other stretchable material. In the illustrated embodiment, the housing 1310 fully conceals the actuator 1320. When the actuator is in a raised position, the telescopic section 1330 forms an exterior or sidewall of the housing 1310. In the illustrated embodiment, the lifting assembly 1200 comprises a pair of telescopic sections 1330, 1230. In the illustrated embodiment, the housing 1310 comprises a rectangular shaped cross section. In the illustrated embodiment, a plate 1390 is disposed at the base of the lifting assembly 1200 to provide a hard, level surface to account for a soft floor surface, such as a rug or carpeting. The plate 1390 comprises a rectangular shape, however, in alternate embodiments, the plate comprises any suitable shape.

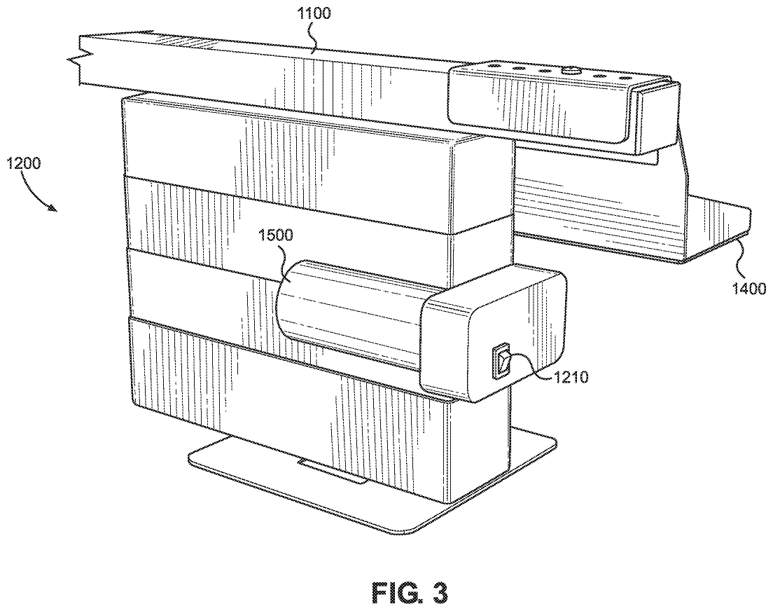

The actuator 1320 is configured to apply a lifting force to the base frame or directly to the bed support, wherein the force is applied when the housing 1310 is moved from the lowered or retracted position to the extended or raised position. As a result, a portion of the force is transferred to the bed frame when moved from a level or floor configuration to an inclined configuration. In the illustrated embodiment, the actuator 1320 is a linear actuator configured to provide a vertical motion. The linear actuator is moved electrically by a motor (see FIG. 3, annotation 1500) disposed exterior to the housing 1310. In other embodiments, the motor is disposed within the housing. In the shown embodiment, the linear actuator comprises a scissor lift 1340 having a first arm 1350 pivotally joined to a second arm 1360 at the distal ends thereof. The motor is operably connected to the scissor lift via a screw 1370 extending from the motor through threaded joints 1380 of the first and second arms 1350, 1360. As the screw 1370 is rotated by the motor, the joints 1380 are forced towards or away from each other resulting in linear movement of the scissor lift. The scissor lift 1340 lowers as the joints are forced away from each other or raises as the joints are forced towards each other.

In the illustrated embodiment, the lifting assembly 1200 is configured to apply varying amounts of torque as the actuator 1310 is raised and lowered. For example, in some embodiments, higher torque is applied when the bed inclining system is raising the bed from the level configuration and the scissor lift is completely compressed, wherein the lifting assembly 1200 transitions to lower torque as the bed continues to move to an inclined configuration and the scissor lift becomes extended. In some embodiments, the varying torque is achieved via different thread configurations disposed on the screw. For example, the screw comprises shallower angles at a distal end, wherein the angles become steeper as the scissor lift is raised.

In some embodiments wherein the bed inclining system comprises multiple lifting assemblies, a first lifting assembly is a master unit configured to operate and/or control activation of a second lifting assembly. The first and second lifting assemblies are wirelessly connected or wired to each other, such that the second lifting assembly is adapted to provide secondary mechanical support. In some embodiments, each lifting assembly comprises a motor.

In other embodiments, the actuator comprises any suitable configuration adapted to raise and lower the housing upon actuation. In some embodiments, the bed inclining system comprises any suitable actuating mechanism, such as a mechanical, a pneumatic or a hydraulic lift mechanism, to facilitate raising and lowering a head of the bed.

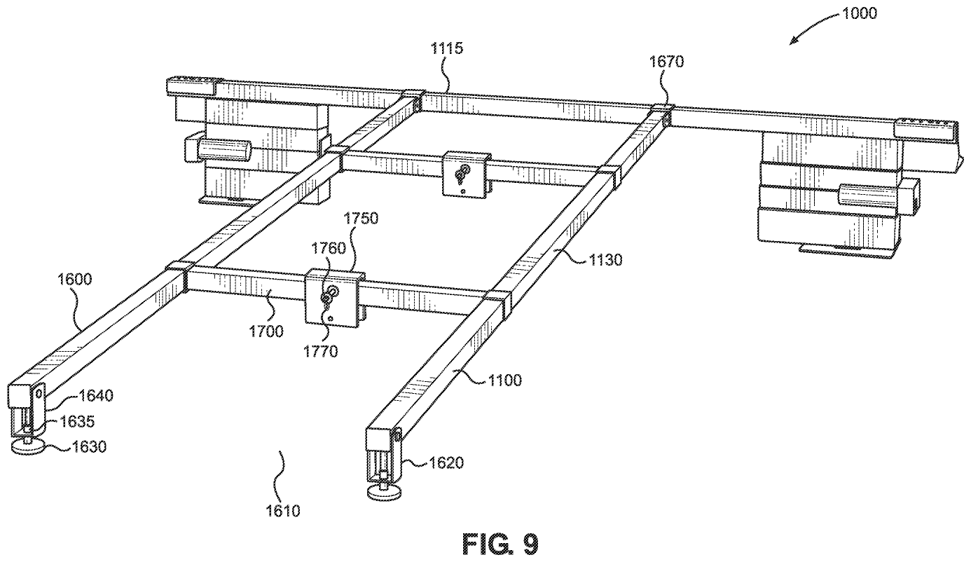

Referring now to FIG. 9, there is shown a perspective view of a third embodiment of the bed inclining system. In the illustrated embodiment, the base frame 1100 of the bed inclining system 1000 comprises a support structure 1130 extending from the bar 1115, wherein the support structure 1130 is adapted to support a mid-section of the bed to provide a planar sleeping surface. In the illustrated embodiment, the support structure 1130 of the base frame 1100 comprises a pair of rod members 1600 extending substantially perpendicular from the elongated bar 1115. The rod members 1600 are substantially parallel to one another and are spaced apart such that a gap 1610 is formed therebetween. In some embodiments, the position of each rod member 1600 along the elongated bar 1115 is adjustable via a bracket fastener 1670, such that the gap 1610 can become wider or smaller. In this way, the placement of the rod members 1600 can be adjusted to align with more vulnerable areas of a bed or a position needing additional support, such as directly beneath a portion of the mattress a user lies upon or beneath a structural element of a box spring or similar structure. In some embodiments, the base frame comprises a single rod member extending from the elongated bar. In other embodiments, the base frame includes more than two rod members. In the illustrated embodiment, the rod members 1600 extend directly from the elongated bar 1115. In the illustrated embodiment, the base frame 1100 comprises only a single elongated bar. In some embodiments, such as the first embodiment shown in FIG. 1, the base frame does not include the support structure. In some embodiments, the support structure is a removable and optional feature to the base frame.

A support foot 1620 is disposed on a distal end of each rod member 1600. Each rod member 1600 is pivotally connected to the support foot 1620 to allow an angle between the rod member 1600 and support foot 1620 to change as the lifting assembly raises and lowers the elongated bar 1115 of the base frame 1100, allowing the support foot to remain flush with a ground surface. In the illustrated embodiment, the support foot is adjustable in height to allow the base frame to engage with beds and bed frames having different dimensions and distances from the ground surface. In the shown embodiment, the support foot 1620 comprises a base 1630 having a screw member 1635 adjustably mounted between a distal end of a bracket 1640. The bracket 1640 extends from the rod member 1600. As the base 1630 is rotated in a first direction, the support foot 1620 is lengthened via the screw member 1635 extending from the bracket 1640 and moving downward. As the base 1630 is rotated in an opposing second direction, the support foot 1620 is shortened via the screw member 1635 retracting into the bracket 1640.

In the illustrated embodiment, the support structure 1130 of the base frame 1100 comprises a pair of rigid crossbars 1700 extending perpendicularly between the pair of rod members 1600. The crossbars 1700 are substantially parallel to the elongated bar 1115 and are configured to provide additional structural support to the support structure 1130 of the base frame 1100. In some embodiments, the base frame 1100 comprises a single crossbar. In alternate embodiments, the support structure does not comprise any crossbars. In some embodiments, the crossbar 1700 is telescopic to permit adjustment of the distance between the pair of rod members 1600.

In the illustrated embodiment, each crossbar 1700 comprises an adjustment member 1750 for adjusting a height such that as the base frame is secured to the bed frame to achieve a planar position. The adjustment member 1750 comprises a U-shaped bracket positioned over a top side of the crossbar 1700. A fastener 1760 extends through an elongated aperture 1770 of the U-shaped bracket, wherein the elongated aperture 1770 permits vertical movement of the U-shaped when the fastener 1760 is loosened or unsecured. Once the U-shaped bracket is at a desired height, the fastener 1760 is secured to prevent movement of the U-shaped bracket.

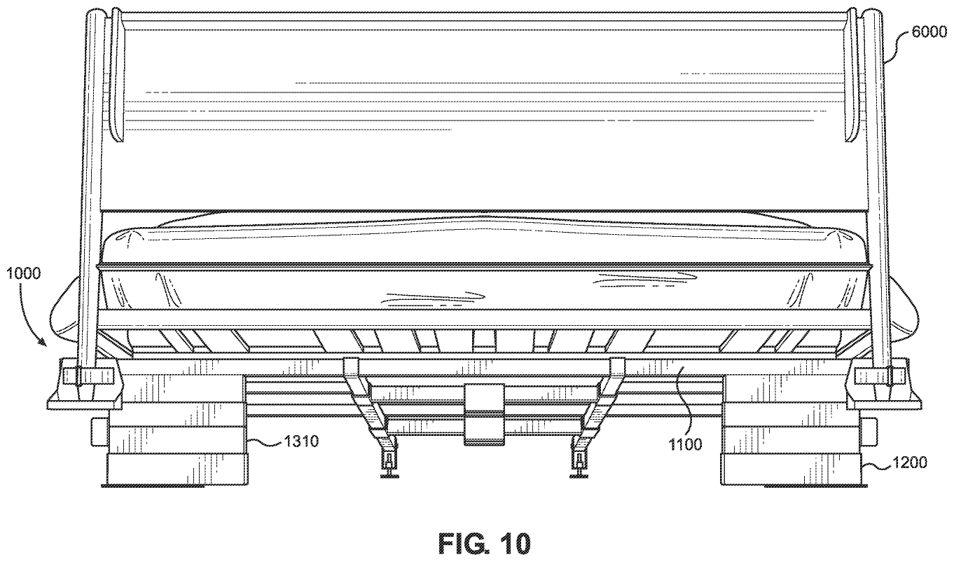

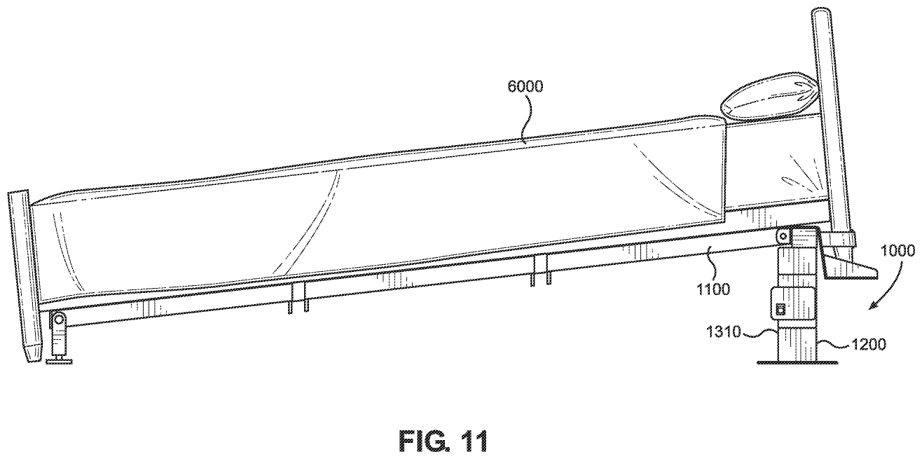

Referring now to FIGS. 10-11, there is shown a rear view of a third embodiment of the bed inclining system in use and a side view of a third embodiment of the bed inclining system in use, respectively. In operation, the elongated bar of the base frame 1100 is positioned at the head of the bed 6000 and the support structure is positioned beneath the bed 6000. In this orientation, the bed supports extend outwards and are configured to receive the legs of the bed. The lifting assembly is positioned beneath the bed, whereas the elongated bar of the base frame 1100 extends substantially between the width of the bed. Once the bed inclining system 1000 is mounted to the bed 6000, the lifting assembly 1200 is activated either remotely or locally. In operation of the first embodiment and the second embodiment shown, only the elongated bar is mounted to the bed.

As a result of the housing 1310 movement via the actuator and the position of the base frame 1100 at the head of the bed, the bed 6000 is configured to be moved between a level configuration and an inclined or raised configuration. In the level configuration, the bed is in a substantially horizontal position, and, in the inclined or raised configuration, the head end or foot end of the bed frame is raised from a floor and is raised relative to the opposite end of the bed frame. The bed support is adapted to support the head of the bed frame in both the level configuration and the raised configuration. In some embodiments, the lifting assembly is directly connected to the bed support such that as the lifting assembly is raised and lowered, so is the bed support.

In the illustrated embodiment, the bed inclining system 1000 is configured to mount to an end (foot or head) of a conventional or pre-existing bed or bed frame. When the bed is in either the level or raised configurations, the opposite end of the pre-existing bed remains on the ground surface and only the end of the bed with the system 1000 mounted thereto is elevated. The bed inclining system 1000 does not bend or in any way angle the pre-existing bed or bed frame, but instead maintains a planar or flat orientation of the bed at all times.

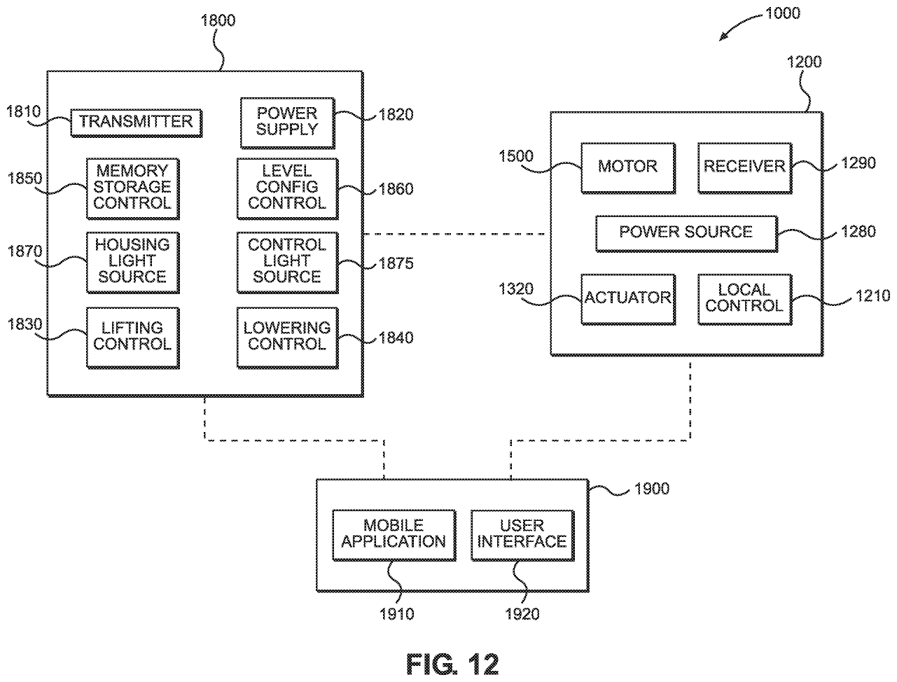

Referring now to FIG. 12, there is shown a block diagram of a control circuit of an embodiment of the bed inclining system. In some embodiments, the bed inclining system 1000 comprises a remote electronic device 1800 configured to activate and control the lifting assembly 1200. In the illustrated embodiment, the remote electronic device 1800 is wirelessly connected to the lifting assembly 1200 and configured, via a transmitter 1810 to communicate commands thereto, via a receiver 1290. The remote electronic device 1800 comprises a power supply 1820, such as a rechargeable battery. In the illustrated embodiment, the lifting assembly 1200 also comprises a power source 1280.

The remote electronic device comprises a manual lifting control 1830 or switch adapted to cause the lifting assembly 1200 to rise so long as the lifting control remains pressed or otherwise activated until the user ceases activation or applying force to the lifting control 1830. The remote electronic device 1800 further comprises a lowering control 1840 or switch that is adapted to cause the lifting assembly 1200 to lower so long as the lowering control remains pressed or otherwise activated until the user ceases activation or applying force to the lowering control 1840.

In some embodiments, the remote electronic device 1800 comprises a memory storage for allowing a user to select pre-saved raised configurations via one or more selectable memory controls. A sensory of the lifting assembly is configured to detect the position and orientation of the bed. Each memory storage control 1850 corresponds to a different elevation of the lifting assembly 1200 selected by the user. Upon finding a desired raised configuration, the user is able to store the desired raised configuration in the memory storage by pressing one of the memory storage controls 1850 in a predetermined memory storage sequence. Thereafter, when that memory control is activated, the lifting assembly 1200 will raise or lower to the configuration associated with the memory storage control 1850. The user can override the selected memory storage by pressing the predetermined sequence again. In some embodiments, the remote electronic device 1800 comprises a planar configuration control 1860 or switch configured to return the lifting assembly 1200 to the lowered position that corresponds to the bed moving to a planar configuration.

In other embodiments, the remote electronic device 1800 comprises a light source to allow a user to locate more easily the remote 1800 in a dark or dimly lit room. In some embodiments, all or parts of the controls or switches of the remote electronic device 1800 are translucent and configured to illuminate when a housing of the remote electronic device 1800 is touched by the user via a sensor disposed within the housing. In other embodiments, the housing is transparent or translucent to allow continuous illumination of the light source. In alternate embodiments, the housing comprises a sensor configured to detect light or lack thereof, such that the light source becomes illuminated upon detection of a room light being turned off. In the illustrated embodiment, the housing light source 1870 is distinct from one or more control light sources 1875.

In some embodiments, the bed inclining system 1000 comprises a secondary remote device 1900, as a smart device, having an application 1910 configured to track user data, such as heart rate, respiration, snoring, and sleep position. In other embodiments, the remote electronic device 1800 comprises the application capable of tracking user data. In some embodiments, the application is operably connected to a wearable device disposed on the user, wherein the wearable device is configured to detect the user data to be collected by the application. The application is configured to automatically adjust a slope of the bed in response to the user data for a better sleep position. In some embodiments, secondary remote device comprises a user interface 1920 that allows a user to pre-program the lifting assembly to automatically adjust to a pre-determined level or raised configuration depending on a time input.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A bed inclining system, comprising:
a base frame configured to be disposed at an end of a bed;
a lifting assembly secured to the base frame, the lifting assembly comprising:
an actuator adapted to raise and lower a bed support, wherein the base frame is configured to be moved between a floor configuration and a raised configuration by the actuator;
wherein the floor configuration, the base frame is configured to position the bed in a substantially horizontal position;
wherein the raised configuration, the base frame is configured to raise the end of the bed from a floor, wherein the end is raised relative to an opposite end of the bed, such that the entire bed is in an inclined position;
wherein the base frame comprises an elongated bar adapted to align the lifting assembly and the bed support in the raised configuration;
wherein the bed support extends from the base frame and is adapted to support the end of the bed in both the floor configuration and the raised configuration.

2. The bed inclining system of claim 1, wherein the bed support is disposed on an exterior side of the base frame and the lifting assembly is disposed on an opposing interior side of the base frame, such that the base frame and lifting assembly are configured to be concealed and positioned beneath the bed when mounted to the bed.

3. The bed inclining system of claim 1, wherein the base frame comprises a first end configured to be positioned at the end of the bed and a second end adapted to support a mid-section of a bed frame to provide a planar sleeping surface.

4. The bed inclining system of claim 1, wherein the bed support comprises a platform having a sidewall disposed along a perimeter thereof, wherein the platform is adapted to receive the leg of the bed frame thereon.

5. The bed inclining system of claim 4, wherein the bed support comprises a fastener adapted to secure the leg of the bed frame to the base frame.

6. The bed inclining system of claim 4, wherein the bed support comprises a pair of bed supports each secured to an opposing end of the base frame and on an opposing side to the lifting assembly.

7. The bed inclining system of claim 6, wherein the platform of each bed support is adjustably mounted to the base frame via a U-shaped plate having a plurality of apertures sized to engage with a corresponding pin of base frame such that a distance between the pair of bed supports is adjustable.

8. The bed inclining system of claim 1, wherein the base frame comprises an elongated bar.

9. The bed inclining system of claim 3, wherein the first end of the base frame comprises an elongated bar and the second end comprises a rod member extending substantially perpendicular from the elongated bar, wherein a support foot is disposed on a distal end of the rod member.

10. The bed inclining system of claim 9, wherein the rod member is pivotally connected to the support foot, wherein the support foot is adjustable in height.

11. The bed inclining system of claim 10, wherein the base frame further comprises a pair of crossbars connecting a pair of rod members, each crossbar comprising an adjustment member for adjusting a height such that as the base frame is secured to the bed frame to achieve a planar position.

12. The bed inclining system of claim 1, wherein the lifting assembly comprises an expandable housing secured to a lower side of the base frame, wherein the actuator is disposed within the housing and adapted to extend and retract the housing.

13. The bed inclining system of claim 12, wherein the housing comprises at least one telescopic section that is retracted within the housing when the actuator is in a lowered position and forms a sidewall of the housing when the actuator is in a raised position.

14. The bed inclining system of claim 1, wherein the lifting assembly comprises a housing mounted centrally on an elongated bar the base frame.

15. The bed inclining system of claim 13, wherein the actuator remains within the housing in both the raised position and a lowered position.

16. The bed inclining system of claim 1, wherein the actuator is in wireless communication with a remote electronic device, the remote electronic device having a control adapted to operate the actuator between a raised position and a lowered position.

17. The bed inclining system of claim 1, further comprising a sensor configured to detect user data from a wearable device, wherein the sensor is operably connected to an application disposed on an electronic device, the application configured to automatically adjust the lifting assembly in response to the user data.

18. A bed inclining system, comprising:
a base frame having first end configured to be disposed at an end of a bed and a second end adapted to support a mid-section of the bed to provide a planar sleeping surface;
wherein the first end comprises an elongated bar and the second end comprises at least one rod member extending perpendicularly to the elongated bar;
a lifting assembly secured to the base frame, the lifting assembly comprising:
an expandable housing and an actuator disposed within the expandable housing;
wherein the actuator is adapted to raise and lower a bed support extending from the elongated bar, wherein the bed frame is configured to be moved between a floor configuration and a raised configuration by the actuator;
wherein the floor configuration, the bed frame is in a substantially horizontal position;
wherein the raised configuration the end of the bed is raised from a floor and is raised relative to an opposite end of the bed;
wherein the bed support extends from the base frame and is adapted to support the head of the bed frame in both the floor configuration and the raised configuration.

19. The bed inclining system of claim 18, wherein the housing comprises at least one telescopic section that is retracted within the housing when the actuator is in the floor configuration and forms a sidewall of the housing when the actuator is in the raised configuration.

20. A bed inclining system, comprising:
a sleeping surface with a head end and a foot end;
a support positioned beneath the head end of the sleeping surface, wherein support is adapted to raise and lower the sleeping surface in response to a user input, causing the sleeping surface to become more or less inclined;
wherein the greater the sleeping surface is inclined, the greater the effects of gravity on a user's circulatory system, digestive system, spine and sinus cavities;
a wireless control device operably connected to the support, wherein the support is configured to receive the user input to increase or decrease the incline of the sleeping surface;
wherein the wireless control device comprises storage of at least one user preference, the at least one user preference corresponding to a specific incline orientation.
